(19) United States
(12) Patent Application Publication
(10) Pub. No.: US 2026/0337687 A1
(43) Pub. Date: Oct. 29, 2026

(54) METHODS FOR ANALYZING EXTRACELLULAR VESICLE-DERIVED DNA MOLECULES IN A SEMEN SAMPLE

(71) Applicant: Fellow Health Inc., San Leandro, CA (US)

(72) Inventors: Kim Michelle Clark-Langone, Redwood City, CA (US); Stephanie Huang, San Carlos, CA (US)

(21) Appl. No.: 19/650,041

(22) Filed: Apr. 16, 2026

Related U.S. Application Data
(60) Provisional application No. 63/796,258, filed on Apr. 28, 2025.

(51) Int. Cl. C12Q 1/6883; C12Q 1/6806; C12Q 1/6886

(52) U.S. Cl. CPC C12Q 1/6883; C12Q 1/6806; C12Q 1/6886; C12Q 2600/154

(57) ABSTRACT

Methods for analyzing high molecular weight exDNA molecules derived from extracellular vesicles (EVs) in a semen sample. The methods may involve, individually or in combination, isolating and analyzing select populations of DNA obtained from somatic cells in a semen sample and size-based sorting. The methods further relate to quantitative analysis of high molecular weight exDNA molecules derived from EVs in a semen sample. The methods described herein also relate to diagnostic methods for detecting prostate cancer, benign prostate hyperplasia (BPH), prostatitis, infertility, or testicular cancer in isolated populations of high molecular weight exDNA molecules derived from extracellular vesicles (EVs) in a semen sample obtained from a patient.

### FIELD OF THE INVENTION

This disclosure relates to methods for analyzing extracellular-derived DNA molecules (e.g., exDNA molecules derived from extracellular vesicles (EVs) released by prostate or testicular cells) in a semen sample. The methods may involve, individually or in combination, isolating and analyzing select populations of exDNA obtained from prostate or testicular cells in a semen sample and size-based sorting. The methods further relate to quantitative analysis of high molecular weight exDNA molecules derived from EVs in a semen sample. This disclosure also relates to diagnostic methods for detecting prostate cancer, benign prostate hyperplasia (BPH), prostatitis, infertility, or testicular cancer in isolated populations of prostate cell-derived exDNA obtained from a patient.

### BACKGROUND OF THE INVENTION

Prostate cancer is one of the most common cancers in the United States, and a leading cause of cancer death in men. Currently, screening for prostate disorders is conducted using blood samples and/or biopsies. Blood samples may be used for testing of Prostate Specific Antigen (PSA) levels in the blood as a biomarker for the detection and monitoring of various disease states. However, issues with this biomarker are known (e.g., over-diagnosis), limiting its accuracy and use in cancer screenings. More recently, assessment of circulating cell free nucleic acids through liquid biopsies is increasingly being used to screen for cancers. Blood samples are a common sample type used for such analysis, however in the prostate cancer context, attempts to detect prostate cancer using circulating tumor DNA in blood have not shown promising results (see, e.g., Lui et al., Ann Oncol. 2020 June; 31 (6): 745-759)). Further, use of liquid biopsies is subject to certain limitations, such as not having sufficient signal and thus introducing a lack of differentiation between a true negative and a false negative. Biopsies are invasive procedures that are cost intensive and/or require recovery time.

Testicular cancer is the most common malignancy affecting young adult men ages 15-40 in the United States (McHugh D J, Gleeson J P, Feldman D R. Testicular cancer in 2023: current status and recent progress. CA Cancer J Clin. 2024; 74 (2): 167-186; Siegel R L, Miller K D, Jemal A. Cancer statistics, 2020. CA Cancer J Clin. 2020; 70 (1): 7-30). While there are no current methods to prevent testicular cancer, early detection plays an important role in increasing survival rates and overall disease prognosis. (Pietrzyk, Ł., Denisow-Pietrzyk, M., Czeczelewski, M. et al. Cancer education matters: a report on testicular cancer knowledge, awareness, and self-examination practice among young Polish men. Sci Rep 10, 20684 (2020)). Globally, male infertility is also on the rise, occurring in around 40% of couples experiencing infertility. (De Jonge, C. and Barratt, C.L.R. (2019)). The present crisis in male reproductive health: an urgent need for a political, social, and research roadmap. (Andrology, 7:762-768). Further, male infertility may be a biomarker for current and future health status of the patient itself. (Chen, Tony et al. Reproductive BioMedicine Online, Volume 44, Issue 1, 131-144).

In view of the foregoing, there remains a critical need in the art to provide a reliable method of isolating DNA of interest (e.g., isolation of DNA from the prostate or testes in a background of DNA from sperm), as well as a way to improve the reliability of non-invasive diagnostic methods including disorder diagnostics relating to the prostate and testes, as well as infertility diagnostics.

### SUMMARY OF THE INVENTION

Aspects of the present disclosure include methods for enriching a semen sample for a plurality of high molecular weight (HMW) exDNA molecules derived from extracellular vesicles (EVs) in the semen sample, the method comprising: centrifuging the semen sample to generate a first pellet and a first supernatant; isolating EVs from the first supernatant; and isolating a DNA fraction from the isolated EVs, comprising a plurality of HMW exDNA molecules derived from EVs, thereby enriching the semen sample for a plurality of HMW exDNA molecules derived from EVs.

Aspects of the invention include methods for analyzing a quantity of HMW exDNA molecules derived from EVs in a semen sample, the method comprising: isolating a DNA fraction comprising a plurality of HMW exDNA molecules derived from EVs of the semen sample; performing an analysis on the DNA fraction to determine an abundance of HMW exDNA molecules derived from EVs in the semen sample; and comparing the abundance of HMW exDNA molecules derived from EVs to a threshold value to determine whether the semen sample contains a sufficient quantity of the HMW exDNA molecules derived from EVs for analysis of a disorder.

Aspects of the invention include diagnostic methods for detecting a disorder from a semen sample, the method comprising: isolating a DNA fraction comprising a plurality of HMW exDNA molecules derived from EVs of the semen sample; performing an analysis on the plurality of HMW exDNA molecules derived from EVs to determine an abundance of HMW exDNA molecules derived from EVs in the semen sample; and comparing the abundance of HMW exDNA molecules derived from EVs to a threshold value to determine whether the semen sample contains a clinically relevant disorder signal.

These and further aspects will be further explained in the rest of the disclosure, including the Examples.

### BRIEF DESCRIPTION OF THE DRAWINGS

FIGS. 1A and 1B are graphs showing the results of cfDNA traces of representative seminal plasma samples from non-vasectomy and vasectomy subjects, respectively.

FIG. 2 is a graph showing the results of a cfDNA trace of a representative seminal plasma sample from a non-vasectomy subject, demonstrating the source of DNA fractions analyzed in the trace.

FIG. 3 is a graph showing the results of an analysis performed to assess loss in exDNA yield after EVs are precipitated and DNase treated, as a function of the amount of granulocyte signal in the seminal plasma of healthy controls prior to EV precipitation.

### DETAILED DESCRIPTION OF THE PREFERRED EMBODIMENTS

Bodily fluids that contain somatic cells and exDNA include blood, urine, saliva, cerebrospinal fluid (CSF) and semen. While much work has been done to study DNA in blood and urine, there has been less work conducted to analyze DNA in CSF, saliva and semen. While semen samples are typically used to study infertility issues, semen samples may also present an alternative method to analyze disorders relating to the prostate and testes, as well as infertility, given the role of the prostate and other reproductive organs in the production of seminal fluid, and the resulting somatic cell-derived DNA (e.g., exDNA and/or DNA from healthy or cancerous somatic cells) present in the seminal fluid.

Previous work has found that significantly more exDNA is present in semen samples obtained from cancer patients as compared with semen samples obtained from healthy patients, however the source of that DNA has not been characterized. (See Ponti et al., Pathol Oncol Res. 2018 October; 24 (4): 941-945) Since DNA extracted from seminal plasma contains both high molecular weight DNA and low molecular weight DNA from both sperm cells and somatic cells, there will be background signal resulting from sperm cell DNA when analyzing signal from somatic cell DNA.

In the same way that white blood cell exDNA impacts the ability to detect tumor exDNA in blood, the background signal from sperm exDNA can impact the ability to detect tumor exDNA (e.g., prostate or testicular cancer) detection in semen. Accordingly, in order to properly analyze signal(s) corresponding to somatic cell-derived exDNA, and specifically, prostate cell-derived exDNA, in a semen sample, background signal(s) corresponding to exDNA derived from sperm cells and other somatic cells in the semen sample must be reduced to improve sensitivity and to yield more accurate results.

Sperm DNA is very tightly packaged into a volume typically 10% or less than that of a somatic cell. This is at least in part due to the fact that histones are largely replaced by smaller, highly basic arginine rich protamines. It is estimated that approximately 85% of sperm DNA is associated with protamines as opposed to histones. (See Bench et al. Cytometry. 1996 April 1; 23 (4): 263-71.) This makes sperm DNA very unique from any other cell type in the human body. Protamine-associated cfDNA derived from sperm cells has a high molecular weight.

Each protamine P1 molecule binds to 10-11 bp of DNA and protamine P2 binds to a slightly larger segment of DNA of approx. 15 bp. (See Balhorn, Genome Biol. 2007; 8 (9): 227.) Protamine molecules bind in the major groove of DNA, neutralizing the phosphodiester backbone of DNA and cause the DNA molecules to coil into toroidal structures, which contain approximately 50,000 bp of DNA (Balhorn, Genome Biol. 2007; 8 (9): 227). Upon sperm cell death, this tight packaging, toroidal structure and protein protection likely makes the DNA resistant to nucleases present in the semen, and likely results in protamine-associated sperm cfDNA being significantly larger than that seen with histone-associated cfDNA.

Additionally, sperm cell structure offers properties that make sperm cells resistant to many of the cell lysis methods that effectively lyse somatic cells. Sperm heads possess a nuclear cap and acrosome which protect the nucleus, and protamines to protect the DNA. Sperm DNA cannot be decondensed in vitro without reducing reagents and sperm nuclei appear to be robust even to sonication (see Perreault et al., J Exp Zool. 1982 Dec. 10; 224 (2): 253-7; and Kuretake et al., Biol Reprod. 1996 October; 55(4): 789-95).

exDNA molecules vary in size ranges depending on their source. As a non-limiting example, exDNA in neutrophil extracellular traps (NETs) may be 1-10 kb, but may degrade to mononucleosome size with time. (See Pisareva et al., Genome Med 14, 135 (2022).) Necrotic cfDNA is generally assumed to be large, typically >10 kb. (See Jahr et al., Cancer Res. 2001 Feb. 15; 61 (4): 1659-65; Rostami et al. Cell Rep. 2020 Jun. 30; 31 (13): 107830).

cfDNA size ranges from 120 bp-220 bp, or multiples thereof, with a peak at 167 bp; this characteristic peak aligns with the length of DNA wrapped around a single nucleosome, plus a short stretch of approx. 20 bp linker DNA bound to a histone (Snyder et al., Cell. 2016 Jan. 14; 164 (1-2): 57-68). Studies have shown that fetal cfDNA is slightly larger, around 143 bp (Jiang et al., Trends Genet. 2016; 32:360-371) and tumor cfDNA slightly shorter, around 150 bp (Lapin et al., J Transl Med. 2018 Nov. 6; 16 (1): 300).

Generally, there is a higher somatic signal in high molecular weight (HMW) cfDNA from seminal plasma than from the nucleosomal fraction of cfDNA. (Huang S, Hart J C, Smith J F, Bench S, Rivas Yepes L, Griscom B, et al. (2025) Tissue of origin characterization of cell free DNA in seminal plasma: Implications for new liquid biopsies. PLOS ONE 21 (3): e0317712.) The source of this HMW somatic cell DNA likely comes from EVs from the epididymis, testes, prostate, kidney, bladder, granulocytes and other white blood cells (e.g., monocytes, T-cells, B-cells, and NK cells). (Aalberts M., et al. Prostasomes: extracellular vesicles from the prostate. Reproduction. 2013, 16; 147 (1); Zijlstra C et al., Prostasomes as a source of diagnostic biomarkers for prostate cancer. J Clin Invest. 2016 Apr. 1; 126 (4): 1144-51; James ER., et al. The Role of the Epididymis and the Contribution of Epididymosomes to Mammalian Reproduction. Int J Mol Sci. 2020 Jul. 29; 21 (15): 5377; Méar L O., et al. Epididymosomes: Composition and Functions for Sperm Maturation. Adv Anat Embryol Cell Biol. 2024 Sep. 24; Smith T I et al., Extracellular vesicles in reproduction and pregnancy. Extracell Vesicles Circ Nucl Acids. 2022 Sep. 30; 3 (3): 292-317.) An additional, potentially abundant source of HMW somatic cell DNA in seminal plasma is from neutrophil extracellular traps (NETs).

Extracellular vesicles harbor various membrane proteins, including, for example without limitation, tetraspanins, adhesion molecules, heat shock proteins, antigen presenting proteins, signaling proteins and growth factors. In the case of prostasomes, membrane proteins include CD9 (Aalberts M., et al. Identification of Distinct Populations of Prostasomes That Differentially Express Prostate Stem Cell Antigen, Annexin A1, and GLIPR 2 in Humans. Biology of Reproduction (2012) 86 (3); 82, 1-8), CD13 (Ronquist G., Prostasomes: Their Characterisation: Implications for Human Reproduction, 2015, Springer International Publishing Switzerlandk The Male Role in Pregnancy Loss and Embryo Implantation Failure, Advances in Experimental Medicine and Biology 868) (Zijlstra C. and Stoorvogel W., Prostasomes as a source of diagnostic biomarkers for prostate cancer, The Journal of Clinical Investigation, Vol. 126, No. 4, April 2016), CD26 (Ronquist 2015) (Zijlstra and Stoorvogel 2016), CD10 (Ronquist 2015) (Zijlstra and Stoorvogel 2016), Annexin 1 (Aalberts et al. 2012) and prostate specific proteins TMPRSS2, prostate-specific transglutaminase, and prostate stem cell antigen (Ronquist 2015) (Aalberts et al. 2012) (Zijlstra and Stoorvogel 2016).

The foregoing discoveries form the foundation upon which the embodiments making up the present application were formed, which are described in greater detail below.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

All references cited throughout the disclosure, including patent applications and publications, are incorporated by reference herein in their entirety.

Recognizing and taking into account the importance and utility of methods that can provide the analyses described herein, the specification and Appendix A describe various embodiments for methods for analyzing somatic cell-derived DNA molecules in a semen sample.

### I. Definitions

For purposes of interpreting this specification, the following definitions will apply and whenever appropriate, terms used in the singular will also include the plural and vice versa. In the event that any definition set forth conflicts with any document incorporated herein by reference, the definition set forth below shall control.

In this application, the use of the singular includes the plural unless specifically stated otherwise. For example, as used in the specification and the appended claims, the singular forms “a,” “an,” and “the” include plural referents unless the context clearly dictates otherwise. Similarly, the adjective “another,” when used to introduce an element, is intended to mean one or more elements. Furthermore, use of the term “including” as well as other forms, such as “include,” “includes,” and “included,” is not limiting.

By “comprising” it is meant that the recited elements are required in the composition/method/kit, but other elements may be included to form the composition/method/kit etc. within the scope of the claim.

By “consisting essentially of,” it is meant a limitation of the scope of composition or method described to the specified materials or steps that do not materially affect the basic and novel characteristic(s) of the subject invention.

By “consisting of,” it is meant the exclusion from the composition, method, or kit of any element, step, or ingredient not specified in the claim.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the phrases “selected from the group consisting of,” “chosen from,” and the like include mixtures of the specified materials.

As described herein, any concentration range, percentage range, ratio range or integer range is to be understood to include the value of any integer within the recited range and, when appropriate, fractions thereof (such as one tenth and one hundredth of an integer), unless otherwise indicated.

Except where expressly noted, trademarks are shown in upper case.

Unless stated otherwise, all percentages, parts, ratios, etc., are by weight.

When an amount, concentration, or other value or parameter is given as a range, or a list of upper and lower values, this is to be understood as specifically disclosing all ranges formed from any pair of any upper and lower range limits, regardless of whether ranges are separately disclosed. Where a range of numerical values is recited herein, unless otherwise stated, the range is intended to include the endpoints thereof, and all integers and fractions within the range. It is not intended that the scope of the present disclosure be limited to the specific values recited when defining a range. Further, where a numerical limit or range is stated herein, the endpoints are included. Also, all values and subranges within a numerical limit or range are specifically included as if explicitly written out.

Further, unless otherwise explicitly stated to the contrary, when one or multiple ranges or lists of items are provided, this is to be understood as explicitly disclosing any single stated value or item in such range or list, and any combination thereof with any other individual value or item in the same or any other list.

As used herein, an “embodiment” means that a particular feature, structure or characteristic is included in at least one or more manifestations, examples, or implementations of this invention. Furthermore, the particular features, structures or characteristics may be combined in any suitable manner, as would be apparent to a person skilled in the art. Combinations of features of different embodiments are all meant to be within the scope of the invention, without the need for explicitly describing every possible permutation by example. Thus, any of the claimed embodiments can be used in any combination.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. When particular values or compositions are provided in the application and claims, unless otherwise stated, the meaning of “about” or “comprising essentially of” should be assumed to be within an acceptable error range for that particular value or composition. When the term “about” is used, it is used to mean a certain effect or result can be obtained within a certain tolerance, and the skilled person knows how to obtain the tolerance. When the term “about” is used in describing a value or an endpoint of a range, the disclosure should be understood to include the specific value or end-point referred to. An exemplary variance of the recited value is 10% of the value.

The terms “exDNA” or “extracellular DNA” refer to DNA that resides outside of cells. ExDNA can either be within extracellular vesicles (EVs), or free from any sort of encapsulation. ExDNA can originate from either passive or active mechanisms. Passive release mechanisms include cell lysis wherein the cell membrane is disrupted, leading to the release of cellular contents (e.g., DNA) and include programmed cell death (apoptosis), necrosis (programmed necrosis), and some forms of NETosis. Active release is when cells secrete exDNA, for example in extracellular vesicles (EVs), which serve a role in cell-to −cell communications, or in some forms of NETosis. The size of exDNA is dependent on its origin (e.g., exDNA resulting from apoptosis is typically around 167 bp and considered low molecular weight, whereas exDNA resulting from necrosis is typically >10,000 bp and considered high molecular weight). exDNA also includes DNA that has been actively secreted from cells, as well as circulating cell-free DNA (cfDNA). cfDNA may result from DNA released by cells following cell death (e.g., cell death occurring due to cell lysis, apoptosis, necrosis, etc.). exDNA derived from sperm cells can be nucleosomal or protamine-associated. exDNA derived from somatic cells is nucleosomal, and does not include protamine-associated exDNA.

The term “prostasome(s)” refers to the extracellular vesicle(s) (EVs) that are released into seminal fluid by prostate cells.

The term “NETosis” refers to the release of a type of exDNA that results in the formation of neutrophil extracellular traps (NETs). NETosis is a process where neutrophils release their DNA as exDNA within a network of extracellular fibers. There are two types of NETosis: suicidal NETosis (i.e., where cells die) and vital NETosis (i.e., where cells remain viable). (See Vorobjeva et al., Biochemistry Moscow 85, 1178-1190 (2020).)

The term “natural cell lysis” means lysis that has occurred due to a biological process and/or lysis that has occurred as a result of environmental conditions (e.g., temperature, pressure, time, etc.) during storage of a sample for collection, transportation, etc.

The term “somatic cell derived DNA” refers to DNA originating from a somatic cell, and specifically includes DNA molecules from a lysed somatic cell (e.g., natural cell lysis, lysis resulting exposure to a lysis reagent, etc.), as well as exDNA molecules originating from a somatic cell.

The term “tissue of origin analysis” refers to a further analysis performed on sequencing analysis results generated for a DNA fraction, in order to determine the proportion(s) of DNA present in the DNA fraction based on tissue cell type.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

### II. Detailed Description

### Separation Techniques to Enrich for Prostate Signal in Seminal Fluid

In one aspect, provided herein is a method for enriching a semen sample for a plurality of high molecular weight (HMW) exDNA molecules derived from extracellular vesicles (EVs) in the semen sample, the method comprising: centrifuging the semen sample to generate a first pellet and a first supernatant; isolating EVs from the first supernatant; and isolating a DNA fraction from the isolated EVs, comprising a plurality of HMW exDNA molecules derived from EVs, thereby enriching the semen sample for a plurality of HMW exDNA molecules derived from EVs.

In one aspect, provided herein is a method for enriching a semen sample for a plurality of high molecular weight (HMW) exDNA molecules derived from extracellular vesicles (EVs) in the semen sample, the method comprising: centrifuging the semen sample to generate a first pellet and a first supernatant; and isolating a DNA fraction from the first supernatant, comprising a plurality of HMW exDNA molecules derived from EVs, thereby enriching the semen sample for a plurality of HMW exDNA molecules derived from EVs. In some embodiments, the methods further comprise a deoxyribonuclease (DNase) treatment step. In some embodiments, the DNase treatment step comprises: treating the first supernatant with a DNase, and incubating the treated first supernatant at 37 °C for 30 minutes.

In some embodiments, centrifuging the semen sample to generate a first pellet and a first supernatant includes centrifuging the semen sample at 16,000 g for ten minutes. In some embodiments, isolating EVs from the first supernatant includes a deoxyribonuclease (DNase) treatment step, a precipitation step, or an antibody enrichment step.

In some embodiments, the deoxyribonuclease (DNase) treatment step comprises: treating the first supernatant with a DNase, and incubating the treated first supernatant at 37 °C for 30 minutes. In some embodiments, the precipitation step comprises: treating the first supernatant with a precipitating reagent to selectively isolate a plurality of exosomes; and centrifuging the treated first supernatant at 1500 g for 30 minutes to generate a second pellet and a second supernatant, wherein the second pellet comprises the isolated EVs. In some embodiments, the second pellet may be resuspended, and treated with a deoxyribonuclease (DNase). In some embodiments, the antibody enrichment step comprises: contacting the first supernatant with one or more extracellular vesicle-specific (EV-specific) immunomagnetic particles to isolate the EVs. In some embodiments, the one or more EV-specific immunomagnetic particles comprise a binding unit that binds to CD9, CD13, CD26, CD10, Annexin 1, prostate specific proteins TMPRSS2, prostate-specific transglutaminase, prostate stem cell antigen, or a combination thereof.

In some embodiments, a size-based separation procedure may be performed on the DNA fraction. Size-based separation procedures are discussed in further detail below. In some embodiments, the methods described herein may additionally include analyzing the DNA fraction comprising a plurality of HMW exDNA molecules derived from EVs through next generation sequencing (NGS) or polymerase chain reaction (PCR).

### Quantitative Analysis of High Molecular Weight exDNA Molecules Derived From Extracellular Vesicles in a Semen Sample

In one aspect, provided herein is a method for analyzing a quantity of HMW exDNA molecules derived from EVs in a semen sample, the method comprising: isolating a DNA fraction comprising a plurality of HMW exDNA molecules derived from EVs of the semen sample; performing an analysis on the DNA fraction to determine an abundance of HMW exDNA molecules derived from EVs in the semen sample; and comparing the abundance of HMW exDNA molecules derived from EVs to a threshold value to determine whether the semen sample contains a sufficient quantity of the HMW exDNA molecules derived from EVs for analysis of a disorder. In some embodiments, the analysis on the DNA fraction is a qualitative analysis. In some embodiments, the analysis on the DNA fraction is a quantitative analysis. In some embodiments, the quantitative analysis is a sequencing analysis. In some embodiments, the sequencing analysis may be performed using any known methods, such as, for example without limitation, a methylation sequencing analysis. In some embodiments, comparing the abundance of HMW exDNA molecules comprises analyzing one or more differentially methylated patterns (DMPs) present in the semen sample.

In some embodiments, isolating the DNA fraction comprising a plurality of HMW exDNA molecules derived from EVs of the semen sample comprises: centrifuging the semen sample to generate a first pellet and a first supernatant; and isolating a DNA fraction from the first supernatant, comprising a plurality of HMW exDNA molecules derived from EVs, thereby enriching the semen sample for a plurality of HMW exDNA molecules derived from EVs. In some embodiments, the methods further comprise a deoxyribonuclease (DNase) treatment step. In some embodiments, the DNase treatment step comprises: treating the first supernatant with a DNase, and incubating the treated first supernatant at 37 °C for 30 minutes.

In some embodiments, isolating the DNA fraction comprising a plurality of HMW exDNA molecules derived from EVs of the semen sample comprises: centrifuging the semen sample to generate a first pellet and a first supernatant; isolating EVs from the first supernatant; and isolating a DNA fraction from the isolated EVs, comprising a plurality of HMW exDNA molecules derived from EVs, thereby enriching the semen sample for a plurality of HMW exDNA molecules derived from EVs. In some embodiments, centrifuging the semen sample to generate a first pellet and a first supernatant comprises centrifuging the semen sample at 16,000 g for ten minutes. In some embodiments, isolating EVs from the first supernatant comprises a deoxyribonuclease (DNase) treatment step, a precipitation step, or an antibody enrichment step.

In some embodiments, the deoxyribonuclease (DNase) treatment step comprises: treating the first supernatant with a DNase, and incubating the treated first supernatant at 37 °C for 30 minutes. In some embodiments, the precipitation step comprises treating the first supernatant with a precipitating reagent to selectively isolate a plurality of exosomes; and centrifuging the treated first supernatant at 1500 g for 30 minutes to generate a second pellet and a second supernatant, wherein the second pellet comprises the isolated EVs. In some embodiments, the second pellet may be resuspended, and treated with a deoxyribonuclease (DNase).

In some embodiments, the antibody enrichment step comprises: contacting the first supernatant with one or more extracellular vesicle-specific (EV-specific) immunomagnetic particles to isolate the EVs. In some embodiments, the one or more EV-specific immunomagnetic particles comprise a binding unit that binds to CD9, CD13, CD26, CD10, Annexin 1, prostate specific proteins TMPRSS2, prostate-specific transglutaminase, prostate stem cell antigen, or a combination thereof.

In some embodiments, the disorder is a prostate-specific disorder. In some embodiments, the prostate-specific disorder is prostate cancer. In some embodiments, the prostate-specific disorder is benign prostatic hyperplasia (BPH). In some embodiments, the prostate-specific disorder is prostatitis. In some embodiments, the disorder is testicular cancer. In some embodiments, the disorder is associated with infertility.

In some embodiments, a size-based separation procedure may be performed on the DNA fraction. Size-based separation procedures are discussed in further detail below. In some embodiments, the methods described herein may additionally include analyzing the DNA fraction comprising a plurality of HMW exDNA molecules derived from EVs through next generation sequencing (NGS) or polymerase chain reaction (PCR).

### Detection of a Disorder Through Analysis of High Molecular Weight exDNA Molecules Derived From Extracellular Vesicles in a Semen Sample

In one aspect, provided herein is a diagnostic method for detecting a disorder from a semen sample, the method comprising: isolating a DNA fraction comprising a plurality of HMW exDNA molecules derived from EVs of the semen sample; performing an analysis on the plurality of HMW exDNA molecules derived from EVs to determine an abundance of HMW exDNA molecules derived from EVs in the semen sample; and comparing the abundance of HMW exDNA molecules derived from EVs to a threshold value to determine whether the semen sample contains a clinically relevant disorder signal. In some embodiments, the analysis on the plurality of HMW exDNA molecules derived from EVs is a qualitative analysis. In some embodiments, the analysis on the plurality of HMW exDNA molecules derived from EVs is a quantitative analysis. In some embodiments, the quantitative analysis is a sequencing analysis. In some embodiments, the sequencing analysis may be a methylation sequencing analysis. In some embodiments, comparing the abundance of HMW exDNA molecules comprises analyzing one or more differentially methylated patterns (DMPs) present in the semen sample. In some embodiments, the sequencing analysis may be performed using any known methods, such as, for example without limitation, a methylation analysis.

In some embodiments, the diagnostic method as described herein may be conducted following identification of a predetermined quantity of HMW exDNA molecules derived from EVs in a semen sample, as described herein.

In some embodiments, isolating the DNA fraction comprising a plurality of HMW exDNA molecules derived from EVs of the semen sample comprises: centrifuging the semen sample to generate a first pellet and a first supernatant; and isolating a DNA fraction from the first supernatant, comprising a plurality of HMW exDNA molecules derived from EVs, thereby enriching the semen sample for a plurality of HMW exDNA molecules derived from EVs. In some embodiments, the methods further comprise a deoxyribonuclease (DNase) treatment step. In some embodiments, the DNase treatment step comprises: treating the first supernatant with a DNase, and incubating the treated first supernatant at 37 °C for 30 minutes.

In some embodiments, isolating the DNA fraction comprising a plurality of HMW exDNA molecules derived from EVs of the semen sample comprises: centrifuging the semen sample to generate a first pellet and a first supernatant; isolating EVs from the first supernatant; and isolating a DNA fraction from the isolated EVs, comprising a plurality of HMW exDNA molecules derived from EVs, thereby enriching the semen sample for a plurality of HMW exDNA molecules derived from EVs.

In some embodiments, centrifuging the semen sample to generate a first pellet and a first supernatant comprises centrifuging the semen sample at 16,000 g for ten minutes. In some embodiments, isolating EVs from the first supernatant comprises a deoxyribonuclease (DNase) treatment step, a precipitation step, or an antibody enrichment step.

In some embodiments, the deoxyribonuclease (DNase) treatment step comprises: treating the first supernatant with a DNase, and incubating the treated first supernatant at 37 °C for 30 minutes. In some embodiments, the precipitation step comprises treating the first supernatant with a precipitating reagent to selectively isolate a plurality of exosomes; and centrifuging the treated first supernatant at 1500 g for 30 minutes to generate a second pellet and a second supernatant, wherein the second pellet comprises the isolated EVs. In some embodiments, the second pellet may be resuspended, and treated with a deoxyribonuclease (DNase).

In some embodiments, the antibody enrichment step comprises: contacting the first supernatant with one or more extracellular vesicle-specific (EV-specific) immunomagnetic particles to isolate the EVs. In some embodiments, the one or more EV-specific immunomagnetic particles comprise a binding unit that binds to CD9, CD13, CD26, CD10, Annexin 1, prostate specific proteins TMPRSS2, prostate-specific transglutaminase, prostate stem cell antigen, or a combination thereof.

In some embodiments, the disorder is a prostate-specific disorder. In some embodiments, the prostate-specific disorder is prostate cancer. In some embodiments, the prostate-specific disorder is benign prostatic hyperplasia (BPH). In some embodiments, the prostate-specific disorder is prostatitis. In some embodiments, the disorder is testicular cancer. In some embodiments, the disorder is associated with infertility.

In some embodiments, a size-based separation procedure may be performed on the DNA fraction. Size-based separation procedures are discussed in further detail below.

In some embodiments, a size-based separation procedure may be performed on the DNA fraction. Size-based separation procedures are discussed in further detail below. In some embodiments, the methods described herein may additionally include analyzing the DNA fraction comprising a plurality of HMW exDNA molecules derived from EVs through next generation sequencing (NGS) or polymerase chain reaction (PCR).

### Size-Based Separation Procedure

In one or more embodiments, methods as described herein further comprise performing a size-based separation procedure on the DNA fraction. In some embodiments, performing the size-based separation procedure comprises separating the DNA fraction into a low molecular weight fraction, a high molecular weight fraction, or a combination thereof. In some embodiments, the low molecular weight fraction comprises molecules (e.g., exDNA, somatic cell derived DNA molecules, or a combination thereof having a size smaller than 500 bp. In some embodiments, the high molecular weight fraction comprises molecules (e.g., exDNA, somatic cell derived DNA molecules, or a combination thereof) having a size greater than or equal to 500 bp.

In some embodiments, a size-based separation procedure is performed on the high molecular weight fraction as described herein, to generate a second low molecular weight fraction comprising molecules (e.g., exDNA, somatic cell derived DNA molecules, or a combination thereof) having a size smaller than 800 bp, and a second high molecular weight fraction comprising molecules (e.g., exDNA, somatic cell derived DNA molecules, or a combination thereof) having a size greater than or equal to 800 bp.

The invention now being fully described, it will be apparent to one of ordinary skill in the art that various changes and modifications can be made without departing from the spirit or scope of the invention.

### Semen Sample

In some embodiments, a semen sample is processed from a patient to generate the seminal plasma sample. In some embodiments, the patient from which the semen sample is obtained has abstained from ejaculation for a time period ranging from 2 to 5 days (e.g., for at least 2 days, for at least 2.5 days, for at least 3 days, for at least 3.5 days, for at least 4 days, for at least 4.5 days, for 2 days, for 3 days, for 4 days, for 5 days, for 2.5 to 4.5 days, or for 3 to 4 days,).

In some embodiments, the semen sample is processed within 12 hours, within 9 hours, within 6 hours, within 5 hours, within 4 hours, within 3 hours, within 2 hours, within 1.5 hours, within 1 hour, within 45 minutes, within 30 minutes, within 15 minutes, within 10 minutes, within 5 minutes, or immediately after/of donation. Between donation and processing, the semen sample is stored at room temperature (i.e., 20-22 °C) or lower, for example at 4 °C to 15 °C, at 4 °C to 12 °C, at 4 °C to 8 °C

In some embodiments, the semen sample may be processed after extended storage at 0 °C or lower, −10 °C or lower, −20 °C or lower, −30 °C or lower, −40 °C or lower, −50 °C or lower, −60 °C or lower, 70 °C or lower, −80 °C or lower. As a lower limit, −90 °C is mentioned. It is further envisioned that the storage temperature may be within a range embraced by any of the foregoing recited extended storage temperatures as the upper limit and -90 °C as the lower limit. Other extended storage temperature ranges are also envisioned including 0 °C to −80 °C, 0 °C to −70 °C, 0 °C to −60 °C, 0 °C to −50 °C, 0 °C to −40 °C, 0 °C to −30 °C, 0 °C to −20 °C, 0 °C to −10 °C, −10 °C to −80 °C, −10 °C to −70 °C, −10 °C to −60 °C, −10 °C to 50 °C, −10 °C to −40 °C, −10 °C to −30 °C, −10 °C to −20 °C, −20 °C to −80 °C, −20 °C to −70 °C, −20 °C to −60 °C, 20 °C to −50 °C, −20 °C to −40 °C, and −20 °C to −30 °C. In some embodiments, when the semen sample is stored at 0 °C or lower, the sample is stored in a semen sample preservative solution (infra) immediately upon collection, such that the sperm cells do not lyse and/or the proteins do not denature. In some embodiments, the semen sample is subjected to a fixation process prior to freezing. In some embodiments, the semen sample may be cryopreserved.

Where the semen sample is stored for an extended period (e.g., a time greater than 12 hours, greater than 18 hours, greater than 24 hours, greater than 36 hours, greater than 48 hours to less than 7 days, less than 6 days, less than 5 days, less than 4 days, less than 3 days) the semen sample may be admixed with a semen sample preservative. Exemplary semen sample preservatives include an antibiotic that prevents microbial growth, cryoprotectants (e.g., dimethyl sulfoxide (DMSO), dimethylformamide (DMF), glycerol, and propylene glycol), formaldehyde, a formaldehyde-derivative, or semen extenders. Exemplary formaldehyde-derivatives include Quaternium-15, DMDM hydantoin, methenamine, methanediol, benzylhemiformal, urea-formaldehyde, urea, paraformaldehyde, pentaerythritol, imidazolidinyl urea, diazolidinyl urea, ethylenediaminetetraacetic acid (EDTA), polyoxymethylene urea, sodium hydroxymethylglycinate, 2-bromo-2-nitropropane-1,3-diol (bronopol), bronidox, and/or glyoxal. In some embodiments, the semen sample is processed by adding preservative immediately upon collection, prior to storing the semen sample. In some embodiments, the seminal fluid is stored for an extended period of time (e.g., a time greater than 12 hours, greater than 18 hours, greater than 24 hours, greater than 36 hours, greater than 48 hours to less than 7 days, less than 6 days, less than 5 days, less than 4 days, less than 3 days), before it is processed into seminal plasma.

Semen extenders include those developed to protect sperm, conserve motility, and fertility over time by stabilizing the plasmalemma, provide energy substrates, and prevent harmful effects of pH and osmolarity changes. The use of semen extenders has the advantage of preserving the sperm in a living state for an extended period, which avoids DNA degradation caused by nucleases present in the semen. Examples of components of semen extenders include low-density lipoprotein and lecithin. In some embodiments, the preservation fluid or sperm preservation fluid may comprise pH stabilizing agents and ions and nutrients to promote sperm health. In some embodiments, the preservation fluid may comprise salts, sugar, and/or an antibiotic agent.

In some embodiments, the semen sample as used in the methods described herein can be contacted with a preservative. In some embodiments, the semen to preservative ratio can range from about 0.1 to about 16 prior to centrifugation.

In some embodiments, the methods described herein can be conducted without freezing the semen sample.

In some embodiments, the methods described herein may additionally include analyzing additional quantities of exDNA and/or cfDNA from the semen sample.

The above written description of the invention provides a manner and process of making and using it such that any person skilled in this art is enabled to make and use the same, this enablement being provided in particular for the subject matter of the appended claims, which make up a part of the original description.

Although this invention has been described with a certain degree of particularity, it is to be understood that the present disclosure has been made only by way of illustration and that numerous changes in the details of construction and arrangement of parts may be resorted to without departing from the spirit and the scope of the invention.

Various modifications to the embodiments will be readily apparent to those skilled in the art, and the generic principles defined herein may be applied to other embodiments and applications without departing from the spirit and scope of the invention. The terminology used herein was chosen to best explain the principles of the embodiments, the practical application or technical improvement over technologies found in the marketplace, or to enable others or ordinary skill in the art to understand the embodiments disclosed herein. Thus, this invention is not intended to be limited to the embodiments shown but is to be accorded the widest scope consistent with the principles and features disclosed herein. It is, therefore, to be understood that within the scope of the accompanying claims, the invention may be practiced otherwise than as specifically described herein.

Having generally described this invention, a further understanding can be obtained by reference to certain specific examples, which are provided herein for purposes of illustration only, and are not intended to be limiting unless otherwise specified.

### Materials and Methods

An IRB approved study, “Fellow Reproductive Improvement Study for Knowledge and Excellence (FRISKE),” enrolled healthy participants to provide fresh semen samples. Preservative was added within 2 hours of collection.

500 ul of preservative was added to 500 ul-6 ml semen, and stored for 2 days. Seminal plasma was prepared by performing a single 16,000 g spin for 10 mins. Extracellular vesicles were obtained by precipitation using the Qiagen™ miRCURY Exosome Serum/Plasma Kit. exDNA was extracted from EVs and the supernatant using the Qiagen™ Circulating Nucleic Acids Extraction kit. The high molecular weight DNA from EVs was subject to sonication using a Covaris™ ultrasonicator, followed by a SPRI clean up using 1.5× ratio of SPRI reagent to sample volume.

### III. Examples

### Example 1: Comparison of exDNA Traces of Representative Seminal Plasma Samples From Non-Vasectomy vs. Vasectomy Subjects

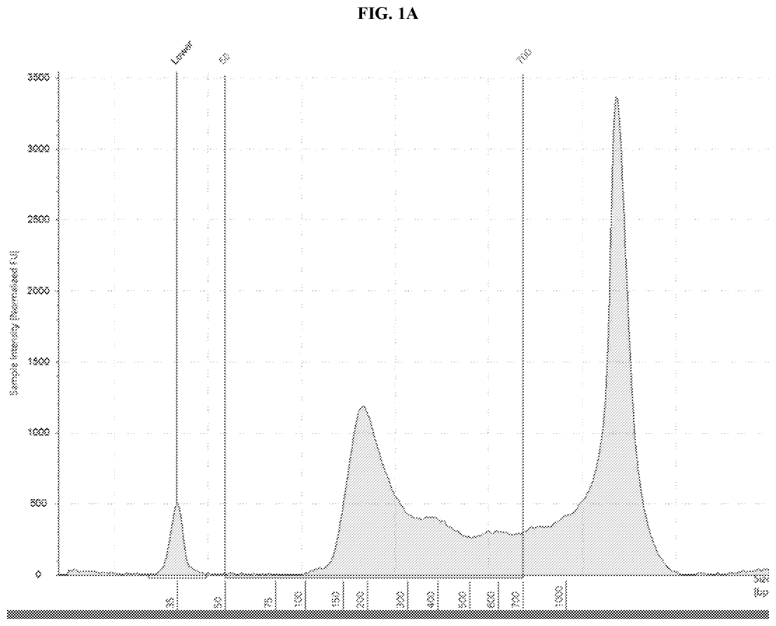

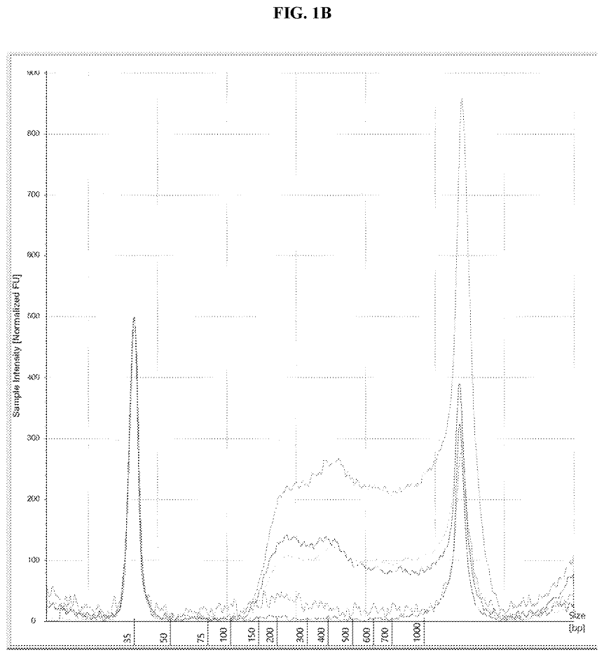

FIG. 1A shows the results of an exDNA trace of a representative seminal plasma sample from a non-vasectomy subject. ExDNA from seminal plasma from men without vasectomy typically contains both nucleosomal DNA (with a peak around 167 bp) and high molecular weight (HMW) DNA (with a peak>1 kb) (FIG. 1A).

In the case of men with vasectomy, as seen in FIG. 1B, which shows the results of ExDNA traces from seminal plasma samples from 5 vasectomy subjects, there is no distinct nucleosomal peak, but rather what appears to be either a complete absence of nucleosomal DNA, or degraded HMW DNA. Similar traces are seen in a subset of men without vasectomy. The absence of a distinct nucleosomal peak in men with vasectomy suggests that the HMW DNA is coming (at least in part) from somatic cells rather than sperm cells.

### Example 2: Results of an exDNA Trace of a Representative Seminal Plasma Sample From a Non-Vasectomy Subject

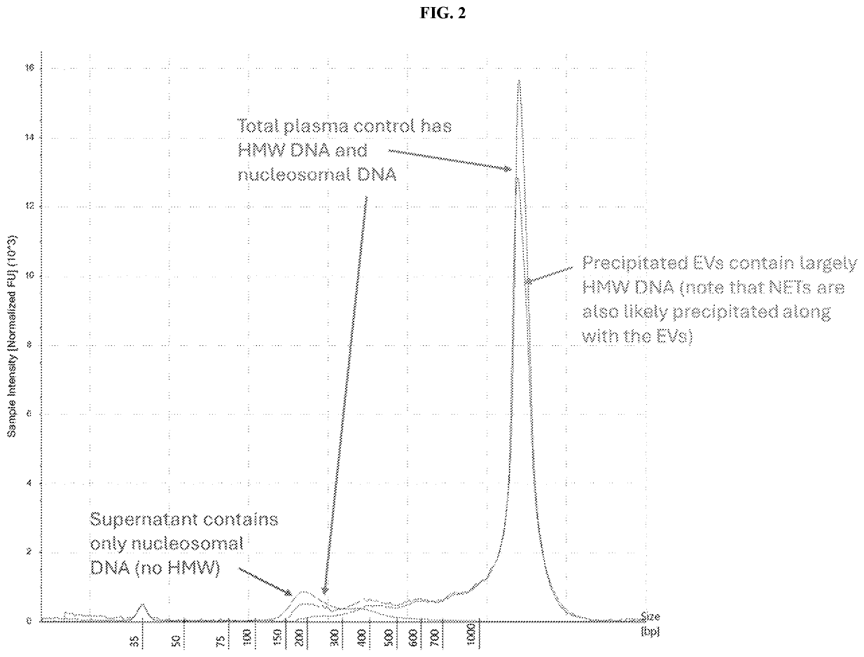

FIG. 2 shows the results of an exDNA trace of a representative seminal plasma sample from a non-vasectomy subject. ExDNA from non-vasectomy seminal plasma typically contains both nucleosomal DNA (peak around 167 bp) and high molecular weight (HMW) DNA (>800 bp). The donor in FIG. 2 has a very small amount of nucleosomal DNA in their seminal plasma (red line). HMW DNA contains a higher level of somatic cell DNA than the nucleosomal fraction. FIG. 2 demonstrates the source of these two DNA fractions.

The extracellular vesicles (EVs) were first isolated by precipitation, followed by centrifugation to pellet. Neutrophil extracellular traps (NETs) are also expected to precipitate and be spun down with the EVs. The blue line in FIG. 2 illustrates that the supernatant contains nucleosomal exDNA and the green line illustrates the HMW DNA extracted from the precipitated EVs. The source of, at least some, HMW DNA in seminal plasma is therefore from EVs and possibly NETs that pellet along with EVs. When the pellet is resuspended and treated with DNase, some DNA is lost, suggesting that not all HMW is within EVs.

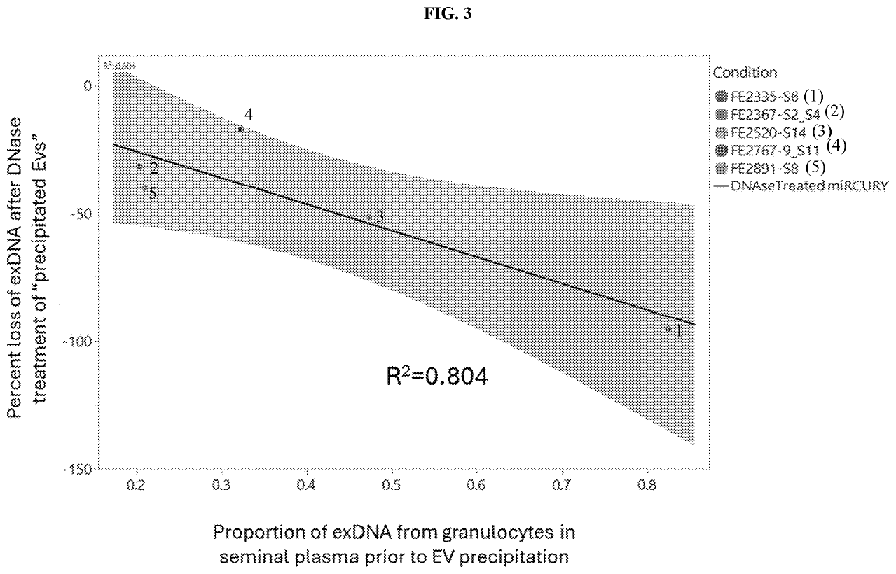

FIG. 3 shows the results of an analysis performed to assess loss in exDNA yield after EVs are precipitated and DNase treated, as a function of the amount of granulocyte signal in the seminal plasma of 5 healthy controls prior to EV precipitation. FIG. 3 illustrates that the amount of DNA that can be depleted is proportional to the amount of granulocyte signal in the sample. Those with higher amounts of granulocyte signal (e.g., FE2335-S6 in FIG. 3) lose more DNA upon DNase treatment than those with low amounts of granulocyte signal (e.g., FE2367-S2_S4 in FIG. 3). As seen in FIG. 3, very little exDNA is lost when precipitated EVs from healthy controls with low granulocyte signal are DNase treated, suggesting the large majority of HMW exDNA is in EVs. However, a large portion of exDNA is lost when precipitated EVs from healthy controls with high granulocyte signal are DNase treated, suggesting the large majority of HMW exDNA that is precipitated is outside of EVs. The exDNA that is precipitated with EVs but disappears upon DNase treatment could represent NETs.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for enriching a semen sample for a plurality of high molecular weight (HMW) exDNA molecules derived from extracellular vesicles (EVs) in the semen sample, the method comprising:
centrifuging the semen sample to generate a first pellet and a first supernatant;
isolating EVs from the first supernatant; and
isolating a DNA fraction from the isolated EVs, comprising a plurality of HMW exDNA molecules derived from EVs, thereby enriching the semen sample for a plurality of HMW exDNA molecules derived from EVs.

2. The method of claim 1, wherein centrifuging the semen sample to generate a first pellet and a first supernatant comprises centrifuging the semen sample at 16,000 g for 10 minutes, and wherein isolating EVs from the first supernatant comprises a deoxyribonuclease (DNase) treatment step, precipitation step, or an antibody enrichment step; and
wherein the antibody enrichment step comprises;
contacting the first supernatant with one or more extracellular vesicle-specific (EV-specific) immunomagnetic particles to isolate the EVs; and
wherein the one or more EV-specific immunomagnetic particles comprise a binding unit that binds to CD9, CD13, CD26, CD10, Annexin 1, prostate specific proteins TMPRSS2, prostate-specific transglutaminase, prostate stem cell antigen, or a combination thereof.

3. (canceled)

4. The method of claim 2, wherein the deoxyribonuclease (DNase) treatment step comprises:
treating the first supernatant with a DNase; and
incubating the treated first supernatant at 37 °C for 30 minutes; and
wherein the precipitation step comprises:
treating the first supernatant with a precipitating reagent to selectively isolate a plurality of exosomes;
centrifuging the treated first supernatant at 1500 g for 30 minutes to generate a second pellet and a second supernatant, wherein the second pellet comprises the isolated EVs; and
further comprising:
resuspending the second pellet; and
treating the resuspended second pellet with a deoxyribonuclease (DNase).

5.-8. (canceled)

9. The method of claim 1, further comprising contacting the semen sample with a preservative, wherein the semen to preservative ratio ranges from about 0.1 to about 16, and wherein the preservative comprises one or more of imidazolidinyl urea, diazolidinyl urea, ethylenediaminetetraacetic acid (EDTA), and an antibiotic that prevents microbial growth.

10.-14. (canceled)

15. The method of claim 1, further comprising performing a size-based separation procedure on the DNA fraction, wherein the size-based separation procedure comprises separating the DNA fraction into a low molecular weight fraction, a high molecular weight fraction, or a combination thereof; and
wherein the low molecular weight fraction comprises exDNA molecules having a size smaller than 500 bp, and wherein the high molecular weight fraction comprises exDNA molecules having a size of greater than or equal to 500 bp; and
performing a size-based separation procedure on the high molecular weight fraction to generate a second low molecular weight fraction comprising exDNA molecules having a size smaller than 800 bp, and a second high molecular weight fraction comprising exDNA molecules having a size greater than or equal to 800 bp.

16.-19. (canceled)

20. The method of claim 1, which is conducted without freezing the semen sample.

21. The method of claim 1, further comprising analyzing the DNA fraction comprising a plurality of HMW exDNA molecules derived from EVs through next generation sequencing (NGS) or polymerase chain reaction (PCR), and analyzing cfDNA from the semen sample.

22.-23. (canceled)

24. A method for analyzing a quantity of HMW exDNA molecules derived from EVs in a semen sample, the method comprising:
isolating a DNA fraction comprising a plurality of HMW exDNA molecules derived from EVs of the semen sample;
performing an analysis on the DNA fraction to determine an abundance of HMW exDNA molecules derived from EVs in the semen sample; and
comparing the abundance of HMW exDNA molecules derived from EVs to a threshold value to determine whether the semen sample contains a sufficient quantity of the HMW exDNA molecules derived from EVs for analysis of a disorder.

25. The method of claim 24, wherein the analysis on the DNA fraction comprises one or more of a qualitative analysis, a quantitative analysis, and a sequencing analysis; and further comprising analyzing cfDNA from the semen sample.

26.-27. (canceled)

28. The method of claim 24, wherein isolating the DNA fraction comprising a plurality of HMW exDNA molecules derived from EVs of the semen sample comprises:
centrifuging the semen sample to generate a first pellet and a first supernatant;
isolating EVs from the first supernatant; and
isolating a DNA fraction from the isolated EVs, comprising a plurality of HMW exDNA molecules derived from EVs, thereby enriching the semen sample for a plurality of HMW exDNA molecules derived from EVs;
wherein centrifuging the semen sample to generate a first pellet and a first supernatant comprises centrifuging the semen sample at 16,000 g for 10 minutes;
wherein isolating EVs from the first supernatant comprises a deoxyribonuclease (DNase) treatment step, a precipitation step, or an antibody enrichment step;
wherein the deoxyribonuclease (DNase) treatment step comprises:
treating the first supernatant with a DNase; and
incubating the treated first supernatant at 37 °C for 30 minutes;
wherein the precipitation step comprises:
treating the first supernatant with a precipitating reagent to selectively isolate a plurality of exosomes; and
centrifuging the treated first supernatant at 1500 g for 30 minutes to generate a second pellet and a second supernatant, wherein the second pellet comprises the isolated EVs; and
wherein the antibody enrichment step comprises:
contacting the first supernatant with one or more extracellular vesicle-specific (EV-specific) immunomagnetic particles to isolate the EVs; and further comprising:
resuspending the second pellet; and
treating the resuspended second pellet with a deoxyribonuclease (DNase).

29.-34. (canceled)

35. The method of claim 34, wherein the one or more EV-specific immunomagnetic particles comprise a binding unit that binds to CD9, CD13, CD26, CD10, Annexin 1, prostate specific proteins TMPRSS2, prostate-specific transglutaminase, prostate stem cell antigen, or a combination thereof.

36. The method of claim 24, wherein the disorder is a prostate-specific disorder, prostate cancer, benign prostatic hyperplasia (BPH), prostatitis, testicular cancer, or a disorder associated with infertility.

37.-41. (canceled)

42. The method of claim 24, further comprising performing a size-based separation procedure on the DNA fraction, wherein the size-based separation procedure comprises separating the DNA fraction into a low molecular weight fraction, a high molecular weight fraction, or a combination thereof;
wherein the low molecular weight fraction comprises somatic cell derived DNA molecules having a size smaller than 500 bp, and wherein the high molecular weight fraction comprises somatic cell derived DNA molecules having a size of greater than or equal to 500 bp; and
performing a size-based separation procedure on the high molecular weight fraction to generate a second low molecular weight fraction comprising somatic cell derived DNA molecules having a size smaller than 800 bp, and a second high molecular weight fraction comprising somatic cell derived DNA molecules having a size greater than or equal to 800 bp.

43.-46. (canceled)

47. The method of claim 24, further comprising contacting the semen sample with a preservative, wherein the preservative comprises one or more of imidazolidinyl urea, diazolidinyl urea, ethylenediaminetetraacetic acid (EDTA), and an antibiotic that prevents microbial growth.

48.-52. (canceled)

53. The method of claim 24, which is conducted without freezing the semen sample.

54. A diagnostic method for detecting a disorder from a semen sample, the method comprising:
isolating a DNA fraction comprising a plurality of HMW exDNA molecules derived from EVs of the semen sample;
performing an analysis on the plurality of HMW exDNA molecules derived from EVs to determine an abundance of HMW exDNA molecules derived from EVs in the semen sample; and
comparing the abundance of HMW exDNA molecules derived from EVs to a threshold value to determine whether the semen sample contains a clinically relevant disorder signal.

55. The diagnostic method of claim 54, wherein the analysis on the plurality of HMW exDNA molecules derived from EVs comprises one or more of a qualitative analysis, a quantitative analysis, and a sequencing analysis;
wherein the sequencing analysis comprises a methylation sequencing analysis, and wherein comparing the abundance of HMW exDNA molecules comprises analyzing one or more differentially methylated patterns (DMPs) present in the semen sample; and
further comprising analyzing cfDNA from the semen sample.

56.-58. (canceled)

59. The diagnostic method of claim 54, wherein isolating the DNA fraction comprising a plurality of HMW exDNA molecules derived from EVs of the semen sample comprises:
centrifuging the semen sample to generate a first pellet and a first supernatant;
isolating EVs from the first supernatant; and
isolating a DNA fraction from the isolated EVs, comprising a plurality of HMW exDNA molecules derived from EVs, thereby enriching the semen sample for a plurality of HMW exDNA molecules derived from EVs;
wherein isolating EVs from the first supernatant comprises a deoxyribonuclease (DNase) step, a precipitation step, or an antibody enrichment step;
wherein the deoxyribonuclease (DNase) treatment step comprises:
treating the first supernatant with a DNase; and
incubating the treated first supernatant at 37 °C for 30 minutes;
wherein the precipitation step comprises:
treating the first supernatant with a precipitating reagent to selectively isolate a plurality of exosomes; and
centrifuging the treated first supernatant at 1500 g for 30 minutes to generate a second pellet and a second supernatant, wherein the second pellet comprises the isolated EVs;
wherein the antibody enrichment step comprises:
contacting the first supernatant with one or more extracellular vesicle-specific (EV-specific) immunomagnetic particles to isolate the EVs; and further comprising:
resuspending the second pellet; and
treating the resuspended second pellet with a deoxyribonuclease (DNase); and
wherein the one or more EV-specific immunomagnetic particles comprise a binding unit that binds to CD9, CD13, CD26, CD10, Annexin 1, prostate specific proteins TMPRSS2, prostate-specific transglutaminase, prostate stem cell antigen, or a combination thereof.

60.-66. (canceled)

67. The diagnostic method of claim 54, wherein the disorder is a prostate-specific disorder, prostate cancer, benign prostatic hyperplasia (BPH), prostatitis, testicular cancer, or a disorder associated with infertility.

68.-72. (canceled)

73. The diagnostic method of claim 54, further comprising performing a size-based separation procedure on the DNA fraction;
wherein performing the size-based separation procedure comprises separating the DNA fraction into a low molecular weight fraction, a high molecular weight fraction, or a combination thereof;
wherein the low molecular weight fraction comprises somatic cell derived DNA molecules having a size smaller than 500 bp, and wherein the high molecular weight fraction comprises somatic cell derived DNA molecules having a size of greater than or equal to 500 bp; and further comprising:
performing a size-based separation procedure on the high molecular weight fraction to generate a second low molecular weight fraction comprising somatic cell derived DNA molecules having a size smaller than 800 bp, and a second high molecular weight fraction comprising somatic cell derived DNA molecules having a size greater than or equal to 800 bp.

74.-77. (canceled)

78. The diagnostic method of claim 54, further comprising contacting the semen sample with a preservative, wherein the preservative comprises one or more of imidazolidinyl urea, diazolidinyl urea, ethylenediaminetetraacetic acid (EDTA), and an antibiotic that prevents microbial growth.

79.-84. (canceled)
